(19) United States
(12) Plant Patent Application Publication
(10) Pub. No.: US 2006/0117432 P1
(43) Pub. Date: Jun. 1, 2006

(54) SUTERA PLANT NAMED 'SUTCATRWHI'

(75) Inventor: Henricus G.W. Stemkens, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/000,577

(22) Filed: Dec. 1, 2004

(51) Int. Cl. A01H 5/00

(52) U.S. Cl. PLT/263

(57) ABSTRACT

A new and distinct variety of Sutera plant particularly distinguished by its white flowers, earliness to flower, thick stems, heat tolerance and a growing habit that is spreading and trailing.

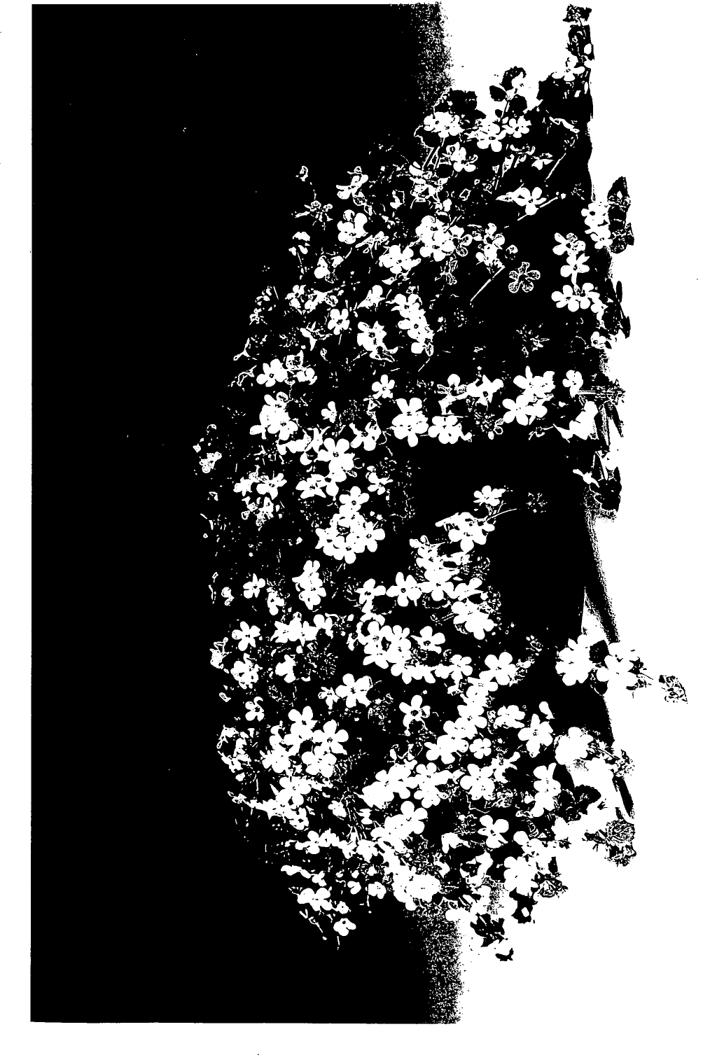

### Latin Name of the Genus and Species of the Plant Claimed

Sutera diffusa

### Varietal Denomination

‘Sutcatrwhi’

### BACKGROUND OF THE NEW PLANT

The present invention comprises a new distinct cultivar of Sutera, botanically known as Sutera diffusa.

The new cultivar is propagated from cuttings resulting from the cross of ‘B22’ and ‘B24’. ‘B22’ is a white flowering Sutera having a spreading, trailing habit. ‘B22’ is not commercially available and is not known by any synonyms. ‘B24’ is a white flowering Sutera having a compact habit. ‘B24’ is not commercially available and is not known by any synonyms. Neither ‘B22’ or ‘B24’ has been patented.

As a result of this cross the present cultivar was created in 2000 in Enkhuizen, Netherlands and has been repeatedly asexually reproduced by cuttings in Enkhuizen, Netherlands and Sarrians, France over a three year period. It has been found to retain its distinctive characteristics through successive propagations, and this novelty appears to be firmly fixed.

This new Sutera plant is an annual in most climatical zones in the US, only in zones 9 and 10 it is a perennial plant.

### SUMMARY OF THE INVENTION

It was found that the cultivar of the present invention:

a) Exhibits white flowers;

b) has thick stems;

c) exhibits a spreading and trailing growth habit; and

d) is very resistant to hot conditions.

### DESCRIPTION OF THE DRAWING

This new Sutera plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Sutera. The data that define these characteristics were collected from asexual reproductions carried out in Enkhuizen, Netherlands. The plant history was taken on 15 weeks old plants, blossomed under natural light in a greenhouse and grown in 11 cm containers. Colour readings were taken in the greenhouse under ambient light. Colour references are primarily to the RHS Colour Chart of The Royal Horticultural Society of London.

TABLE 1
Differences between the new cultivar ‘Sutcatrwhi’,
its parents and a similar cultivar

| | ‘Sutcatrwhi’ | ‘B22’ | ‘B24’ | ‘Bridal Showers’ |
|---|---|---|---|---|
| Flower color | N155A | 155C | 155B | 155C |
| Stem thickness | Thick | Medium | Thick | Thin |
| Plant habit | Trailing | Trailing | Compact | Trailing |
| Flower size | Medium | Big | Small | Small |

‘Bridal Showers’ is not patented.

### The Plant

Classification — Botanical: Sutera diffusa
Parentage — Female parent: A seedling named ‘B22’ is one of our seedlings from our B-generation of plants bred in 1999. — Pollen parent : A seedling named ‘B24’ is one of our seedings from our B-generation of plants bred in 1999.
Growth habit: Spreading and trailing, decumbent.
Plant height: 14-18 cm.
Spreadingarea of plant 40-58 cm.
Growth rate: Rapid growing.
Strength: Good.
Branching habit: Short internodes and on each knot more than two apprearing shoots give a plant that is more branched than normal.
Blooming period: From May until November.

### The Stem

Diameter: 2-3 mm.
Shape: Cylindrical.
Anthocyanin pigmentation: Absent.
Color of the stem: 137B.
Length of internode: 14-28 mm, depending on the light where the plant is propagated.
Pubescence: Sparse.

### The Foliage

Phyllotaxis: Opposite, decussate.
Shape of blade: Broadly ovate.
Shape of the blade apex: Acute.
Shape of the blade base: Truncate.
Leaf margin: Dentate.
Venation: Pinnate.
Texture — Upper surface: Smooth, glabrous — Lower surface: Slightly pubescent.
Length: 14-20 mm.
Width: 14-24 mm.
Thickness: 1-2 mm.
Depth of incision: Less than 1 mm.
Color — Upper side N138B — Lower side: 137C.
Length ofpetiole 1-2 mm.
Diameter of petiole 1 mm.
Color of petiole: 137B.
Fragrance: Musk.

### The Bud

Pedicel length: 8-11 mm, depending on season.
Pedicel diameter: 1 mm.
Pedicel color 137D.
Pedicel angle: About 60 degrees from the stem.
Bud size — Diameter: 1 mm — Length: 4-8 mm.
Bud shape: Obovate.
Bud color: 155A.
Sepals — Color: 137D — Number: 5 — Shape: Erect calyx — Size: 3mm.
Shape of sepals: Gamopetalous, grown together at the base of the sepal.

### The Flower

Flower diameter: 15-18 mm.
Flower tube length: 5-8 mm.
Throat diameter, distal end: 2-4 mm.
Tube diameter, proximal end: 1.5-2.5 mm.
Form: Salverform.
Symmetry: Radial symmetric.
Type: Solitary, axillary.
Color — Upper surface: Between N155A — Lower surface: N155A.
Corolla tube: Cylindrical.
Corolla tube color: 8C.
Overlapping of petals: Separate.
No. of petals: 5.
Shape of the petals: Gamopetalous, grown together at the base of the petal, forming a corolla.
Petal lobe — Length: 4-6 mm — Width: 2-4 mm — Shape: Spatulate — Apex: Rounded — Margin: Entire — Texture: Smooth.
Calyx — Length: 6-8 mm — Diameter 3 mm — Shape: Narrow, linear — Apex: Acute — Margin: Entire.
Texture of the calyx — Upper surface: Pubescent — Lower surface: Pubescent.
Color of the calyx — Upper surface: 137B — Lower surface: 137C.
Anthocyanin pigmentation of calyx limb: Absent.
Flower fragrance: No fragrance detected.
Lastingness of one flower: Flowers stay open 6-8 days.

### Reproductive Organs

Number of reproductive organs: 1 pistil, 4 stamens.
Pollen production: Very low.
Stigma color: 138D.
Stamen color: 13B.
Stamen arrangement: Didynamous.
Stamen type: Basifixed.
Dehiscence of stamens: Extrorse.
Ovary position: Ovary superior, flower hypogynous.
Style shape: Style terete.
Style length: 9 mm.
Pistil length: 12 mm.

### Seeds

No seedset is observed.

### Roots

Type of roots: Fibrous, fine, color 155A.

### Physiological and Ecological Characteristics

Extreme tolerance to heat is observed.

### Propagation

Propagation is done by axillary cuttings. Tips of stems are used, which take 6 days to initiate roots and 12 days to develop roots.

What is claimed is:

1. A new and distinct variety of Sutera plant named ‘Sutcatrwhi’, substantially as illustrated and described herein.
